(19) United States
(12) Patent Application Publication
(10) Pub. No.: US 2025/0318768 A1
(43) Pub. Date: Oct. 16, 2025

(54) SYSTEM AND METHODS FOR PREDICTING BEHAVIOURAL PERFORMANCE OF A SPECIAL-NEED STUDENT USING ARTIFICIAL INTELLIGENCE

(71) Applicant: CENTRE FOR PERCEPTUAL AND INTERACTIVE INTELLIGENCE (CPII) LIMITED, Pak Shek Kok (HK)

(72) Inventor: Yuen Yan CHAN, Pak Shek Kok (HK)

(21) Appl. No.: 18/989,406

(22) Filed: Dec. 20, 2024

Related U.S. Application Data
(60) Provisional application No. 63/632,610, filed on Apr. 11, 2024.

(51) Int. Cl. A61B 5/16; A61B 5/00; A61B 5/0205

(52) U.S. Cl. CPC A61B 5/165; A61B 5/0022; A61B 5/0205; A61B 5/7267; A61B 5/7275; A61B 2503/06

(57) ABSTRACT

Disclosed is a system and methods utilizing artificial intelligence to predict the behavioral performance of special-need students. Ther system has one or more processors connected to a cloud server, which houses multiple programmable modules. The first module is designed to receive raw data from various sensing devices, enabling comprehensive data collection. The second module pre-processes this multimodal data, generating a joint data representation vector within a defined time window. The third module employs an optimized machine learning algorithm to analyze this data and predict the student's performance. This predictive capability offers personalized insights into the student's learning performance, engagement levels, and specific adaptation needs. By leveraging real-time data and advanced analytical techniques, the system aims to enhance educational outcomes and provide targeted support for students with special educational needs.

### CROSS-REFERENCE TO RELATED APPLICATIONS

This application claims priority to U.S. Provisional Patent Application No. 63/632,610, “Multimodal and Individualised Predictive Modelling for Students with Special Education Needs,” filed on Apr. 11, 2024, presently pending.

### FIELD OF THE INVENTION

The present invention relates to a system and method for predicting behavioural performance of a special-need student using artificial intelligence. More specifically, the present invention integrates a framework with theory-grounded artificial intelligence (AI) to provide personalized insights into the student's learning performance, engagement, and adaptation needs.

### BACKGROUND OF THE INVENTION

Students with special education needs (SEN) have disadvantages in aspects such as physical, intellectual, and communication capacities. They face a variety of challenges that can impact their academic and social development. According to the latest available government statistics, the percentage of students with SEN is 17.3% in the UK and 14.7% in the US. These figures suggest that SEN is a non-negligible social and global concern. Students having SEN require additional support to succeed in their learning and development. For example, adaptive pedagogical methods and individualized support are necessary to facilitate their participation in educational programs. It is crucial for the AI community to recognize these exceptional students' needs and address their challenges, so they have equal access to education and are given the opportunity to reach their full potential.

In recent years, AI has been piloted in SEN interventions such as ABA therapy to support the delivery of individualized services. ABA is a well-established approach to behavior modification for students with SEN e.g., autism spectrum disorder (ASD). It is systematic, evidence-based, and data-driven. Meanwhile, predictive modelling (PM) employs existing data and machine learning (ML) techniques to build statistical models for future event predictions. In humanitarian application areas such as healthcare and special education, predictive models' performances have been shown to be further enhanced by the inclusion of theory-supported predictor variables. However, such data often exist across heterogeneous modalities and require non-trivial multimodal ML processing. This makes PM both promising and challenging in social impact domains.

AI for Special Education is an early market with a dearth of available technologies. Previous research involved the collection of multimodal data, and a recommendation of applied behavior analysis (ABA) tasks based on students' educational data. However, the above prior work lacks real-time processing and analysis and considering that the environmental and physiological data are not collected, they may not be able to predict students' behaviors accurately.

In addition to the above, there are a few other available technologies for individualized predictive modelling (PM) in special education, yet, only Rudovic et al. (2018) targets engagement levels of students with autism special disorders (ASD), and supports personalized and multimodal PM at an accuracy of 60.7%. While Daniels et al. (2018) targets the emotions of students with ASD and Huynh-Cam et al. (2022) targets the academic performance of students with general learning disabilities only personalized PM is achieve, and Zahid et al. (2022) targets sign language in deaf students but achieves multimodalities only. The above technologies still lack the capability to process and analyze data in real-time.

An example of an existing technology employing predictive modelling is disclosed in WO2019045637A2, wherein said technology is used in providing personalized clinical decision-support for patients in a hospital to facilitate and improve treatment decisions and patient care management. The multimodal patient data collected in said technology may include physiological data, administrative data, billing data, medical history, admitting characteristics, inpatient care data, etc. and no ABA markers are adopted. Whilst said technology has a machine-learning capability, it may not be applicable for the prediction of SEN students' behavior accurately, as it lacks predictor variables backed up by theories and personalized data such as data from therapy sessions.

US2020234606A1 disclosed computer program products, and systems for collecting instances of user learning data and user personal data for a user and analyzing for common attributes to recommend a course based on the user learning profile, user-topic preferences, and contents of an educational knowledge base. However, said technology lacks personalization and collection of multimodal data that are useful in developing an individualized predictive model to predict behaviors of an SEN student. Furthermore, it also lacks accuracy as it may not have the capability to predict any missing data values in the system.

Some of the most recent Chinese inventions i.e., CN109214664B and CN116049557A may have included machine learning in their inventions, however CN109214664B only employs the use of a single data information and CN116049557A employs multimodal data information. However, both still lack individualization and accuracy in providing a reliable predictive learning model for SEN students.

Lastly, US20220327809A1 may have disclosed a method of training an AI model based on multimodal data collection, however it lacks individualization and accuracy in providing a reliable predictive learning model for SEN students.

In view of the above, there is an unmet need for SEN students to access a platform to incorporate a theory-grounded artificial intelligence (AI) approach in special education contexts. There is also a need for a platform containing real-time data to provide real-time data processing and analysis and an individualized prediction system to allow SEN students to embrace learning intuitively.

### SUMMARY OF THE INVENTION

It is an objective of the present invention to provide an individualized multimodal predictive modelling for special education.

Another objective of the present invention is to provide an accurate learning platform for SEN students with personalized prediction on SEN students' behaviors with real-time processing and analysis.

Generally, the present invention relates to a system for predicting behavioral performance of a special-need student using artificial intelligence comprising: one or more processors; a cloud server coupled to the one or more processors, wherein the cloud server comprises a plurality of building blocks comprising: a first building block configured to receive raw multimodal data from a plurality of sensing devices; a second building block configured to pre-process the collected multimodal data and to produce a joint data representation vector over a defined time window; and, a third building block configured to predict the performance of the special-need student using an optimized machine learning module; wherein, the plurality of building blocks is in the form of programmable instructions executable by the one or more processors.

Additionally, the present invention also teaches a method for predicting behavioral performance of a special-need student using artificial intelligence, the method comprising: collecting real-time multimodal data; pre-processing the collected real-time multimodal data via a multimodal data fusion module and producing a joint data representation vector over a defined time window; and, predicting the behavioral performance of the special-need student using a machine learning module.

The present invention also teaches a method of training and optimizing a machine learning module for predicting behavioral performance of a special-need student comprising: collecting multimodal data via a multimodal data collection module as a training dataset; storing the training dataset in a cloud server; initiating the machine learning module; selecting random samples from the training dataset; computing data loss and updating the machine learning module's parameters by minimizing the data loss; and; repeating steps (d)-(e) until convergence is achieved or a pre-determined upper limit of loops number is reached.

### BRIEF DESCRIPTION OF DRAWINGS

The features of the invention will be more readily understood and appreciated from the following detailed description when read in conjunction with the accompanying drawings of the preferred embodiment of the present invention, in which:

FIG. 1 illustrates an overview of the architecture of the present invention.

FIG. 2 illustrates an overall workflow of the embodiments in the present invention.

FIG. 3 illustrates an IoT sensor box developed in the present invention.

FIG. 4 illustrates a neural network architecture for individualized multimodal predictive modelling.

FIG. 5 illustrates a Scatter Plot of Precision vs. Recall for Baseline Models and the Full Model.

### DETAILED DESCRIPTION OF THE INVENTION

For the purposes of promoting and understanding the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

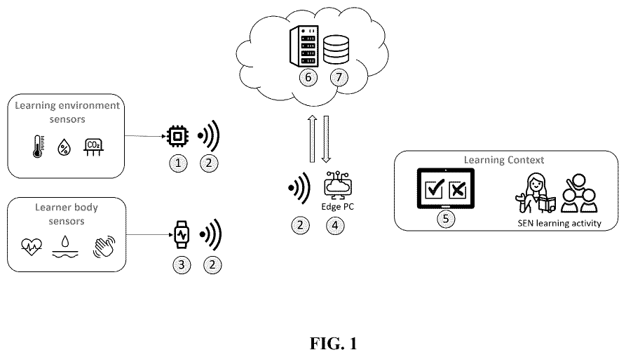

The present invention provides a system for predicting behavioral performance of a special-need student using artificial intelligence comprising: one or more processors; a cloud server coupled to the one or more processors, wherein the cloud server comprises a plurality of building blocks comprising: a first building block configured to receive raw multimodal data from a plurality of sensing devices; a second building block configured to pre-process the collected multimodal data and produce a joint data representation vector over a defined time window; a third building block configured to predict the performance of the special-need student using an optimized machine learning module, wherein the plurality of building blocks is in the form of programmable instructions executable by the one or more processors. This is further illustrated in FIG. 1.

In one embodiment of the present invention, the first building block is a multimodal data collection module.

In one embodiment of the present invention, the second building block is a multimodal data fusion module.

In one embodiment of the present invention, the multimodal data fusion module further comprises: a multimodal translation module that employs temporal information in the multimodal raw data to translate and predict missing data values; a multimodal data alignment module that performs temporal alignment and, algorithmically aligns the translated data based on the defined time window to produce a unified dataset; and, a deep neural network (DNN) joint representation module that employs the unified dataset to produce a joint data representation vector for subsequent analysis.

In one embodiment of the present invention, the third building block is a machine learning module, configured to train, cross-validate, test and predict the behavioral performance of the special-need student. The outcome from the prediction will p provide personalized insights into the student's learning performance, engagement, and adaptation needs.

In one embodiment of the present invention, the collected multimodal data are e student's individualized categorical variables that uniquely couple with student's special needs (SEN) data; classroom environment data; physiological data; and motion data.

In one embodiment of the present invention, the plurality of the sensing devices comprises: an IoT sensor box; and a plurality of sensors includes a temperature sensor, a humidity sensor, and a CO₂ sensor; wherein, each of the plurality of sensors is operatively connected to the IoT sensor box to transmit data to the second building block.

In one embodiment of the present invention, the plurality of the sensing devices further comprises wearable sensors configured to measure heart rate, sweat, and motion.

In one embodiment of the present invention, the unified dataset permits alignment and fusion of multimodal data across different time windows. More specifically, such alignment ensures accurate contextual representation of the student's psychological state, physiological states, and learning environment.

In one embodiment of the present invention, the one or more processors is at least one computing device with internet access, which includes but is not limited to an Edge PC, a tablet, and the like.

In one embodiment of the present invention, the cloud server is configured to store and process multimodal data for model refinement and continuous learning through a feedback module.

Additionally, the present invention also teaches a method for predicting behavioral performance of a special-need student using artificial intelligence, the method comprising: collecting real-time multimodal data; pre-processing the collected real-time multimodal data via a multimodal data fusion module and producing a joint data representation vector over a defined time window; and predicting the behavioral performance of the special-need student using a machine learning module.

In one embodiment of the present invention, the step of collecting the real-time multimodal data further comprises: placing a plurality of sensing devices on the student and the student's classroom environment; capturing the student's classroom environment data, physiological data, and motion data; and, transmitting the real-time multimodal data via a wireless connection to a multimodal data collection module.

In one embodiment of the present invention, the step of pre-processing the collected multimodal data further comprises: retrieving existing SEN data; translating the captured data via a multimodal translation module; formulating an embedding vector for the SEN data; aligning the multimodal data to ensure timestamp consistency between each data collected via a multimodal data alignment module; providing at least one stimulus and prompt using an assessment marker by a human expert via a computing device and automatically tagging a timestamp to each data inputted by the human expert; formulating an input vector by combining the embedding vector for the SEN data and translated data; and, applying a deep neural network (DNN) to the input vector, producing the joint data representation vector, and, projecting it into a multimodal space for subsequent analysis.

In one embodiment of the present invention, the step of predicting the behavioral performance of the special-need student in real-time using the machine learning module further comprises: feeding the predicted behavioral performance back to the machine learning module via a feedback module. Consequently, the feedback module improves the accuracy of the machine learning module in predicting future behavioral performance of the student.

In one embodiment of the present invention, the step of translating the captured data via a multimodal translation module further comprising: creating a multi-modality and multi-temporal sensing dataset; containing time stances of the student's SEN data collected in a session; and predicting missing sensor data in the session.

In one embodiment of the present invention, the step of aligning the multimodal data to ensure timestamps consistency between each data collected via a multimodal data alignment module further comprising: synchronizing unimodal measurements; and, producing a single and coherent dataset

In one embodiment of the present invention, the step of collecting the multimodal data further comprising: selecting at least one SEN data from a group of behavioral tasks including academic and learning tasks, behavior development, communication, independence and self-help, sensory-motor skills and socio-emotional skills.

In one embodiment of the present invention, the step of collecting the multimodal data further comprising selecting the student being diagnosed with learning disabilities including but not limited to mild to moderate autism spectrum disorder (ASD) or intellectual disabilities.

In one embodiment of the present invention, the step of providing at least one stimulus and the prompt using the assessment marker by the human expert via the computing device and automatically tagging the timestamp to each data inputted by the human expert further comprising: continuing or pausing the step of providing the at least one stimulus and the prompt according to a student's condition based on the data captured by the plurality of the sensing devices; and, repeating the step of providing the at least one stimulus and the prompt until the student provides a correct response or session ends.

In one embodiment of the present invention, the step of applying the deep neural network (DNN) to the input vector, producing the joint data representation vector and projecting into the multimodal space for subsequent analysis by the machine learning module further comprising: processing the input vector through hidden layers of the DNN; utilizing a penultimate layer of the DNN; and, applying an output activation function to the penultimate layer to map the joint to output the vector.

In one embodiment of the present invention, the step of predicting the behavioral performance of the special-need student in real-time using the machine learning module further comprising: utilizing the joint data representation vector to perform the behavioral prediction of the performance the special-need student.

Lastly, the present invention also teaches a method of training and optimizing a machine learning module for predicting behavioral performance of a special-need student comprising: (a) collecting multimodal data via a multimodal data collection module as a training dataset; (b) storing the training dataset in a cloud server; (c) initiating the machine learning module; (d) selecting random samples from the training dataset; (e) computing data loss and updating the machine learning module's parameters by minimizing the data loss; and; (f) repeating steps (d)-(e) until convergence is achieved or a predetermined upper limit of loops number is reached.

In one embodiment of the present invention, the method further comprising: formulating a joint data representation vector from a real-time measurement; and, making a prediction.

In summary, the goal of the present invention is to enhance AI's usage in special education using domain knowledge and experience in the field. The MIPM is a theory-grounded AI approach applicable to special education. The main contributions include a novel MIPM as an end-to-end machine-learning (ML) framework for personalized learning in special education. There is empirical evidence that MIPM statistically significantly improves over baseline models with either personalized or multimodal predictors alone. Rigorous experimentation of MIPM using authentic multimodal data has been conducted to evaluate the performance and effectiveness of this approach.

Applied Behavior Analysis (ABA) is an intervention approach in special education. It aims at promoting behaviors that are important for students' social functioning and reducing problematic behaviors. ABA employs pedagogical strategies rooted in (1) Skinner's behaviorism and (2) behavior analysis. Behaviorism is an educational paradigm that views learning as the modification of an organism's behavior and explains learning in terms of a function of environmental factors. Behavior analysis is a scientific approach to studying human and animal behavior through experimentation, such as controlling and changing the factors that affect the behaviors being analyzed. ABA views behavior as the learner's interaction with the surrounding environment, while learning is influenced by the entire set of physical circumstances in which the learner is. Principles and methods of the science of behavior are applied in ABA to improve behavior in practical, real-world settings.

Multimodal Factors Affecting SEN Student Behavior and Learning Educational Factors Educational factors refer to traditional variables associated with students learning. In the SEN context, this includes whether the student has access to an adaptive curriculum and whether an inclusive school environment is being provided. It is known that an increase in access to inclusive environments and individualized curricula can promote learning for students with SEN. Environmental Factors Students with SEN often have impairments in sensory processing and are sensitive to the surrounding environment. For example, high levels of CO₂ content can easily cause fatigue and distractibility in students with SEN. High ambient temperature and acoustic discomforts are found to affect mood and cause distraction in them, too.

Besides, they are easily affected by inappropriate lighting and glare in classrooms. Physiological Factors Research has shown that abnormal levels of skin conductance (measured through galvanic skin response, GSR), whether too high or too low, can impede the learning performance of students with SEN. It is also shown that body movement can positively impact SEN students' short-term memory functioning. Besides, skin temperature is found to be a positive predictor of SEN students' behavioral learning in a recent study.

Learning analytics (LA) encompasses collecting, analyzing, and using data related to learners and learning contexts to optimize the learning processes and environments. Multimodal learning analytics (MMLA) further enhances LA by employing additional educational data in multiple modalities such as texts (e.g., activity logs), audio, video, and sensors. Internet of Things (IoT) devices and sensors have enabled the capturing of educational data in ways and formats not possible before. They enhance MMLA studies that pertain to a variety of learning environments and outcomes for students with SEN. According to a recent systematic review, IoT sensors had been used to capture data in special education domains, including (1) students' bodily movement (such as head, hands, and body), (2) physiological conditions (such as blood volume pulse, skin conductance, and skin surface temperature), and (3) the ambient learning environment (such as light intensity, humidity, temperature). A few examples also include the estimation of the engagement during robot-assisted therapy for children with autism and the evaluation of SEN students' academic performance based on multimodal data collected from educational games.

AI research has begun to pertain to special education. However, only a limited number of existing works are supported by empirical performance evaluation. These include a deep model taking multimodal data developed to learn the engagement of children with ASD, an ML model applied to assist children with autism in recognizing emotions during social interactions and ML models constructed to predict the academic performance of university students with learning disabilities (LD). Besides, computer vision and natural language processing are used to recognize sign language for those with speech impairment. There is a wide variation in skills and clinical conditions among students with SEN, and it is important to provide personalized learning opportunities to tailor students' needs. However, not many AI for special education tools developed support personalization. In view of the above, the MIPM machine-learning framework extends the existing practices in two ways:
Individualization: Anonymized personalized educational data are utilized to make student-centered predictions while maintaining user privacy at the edge-layer level.
Multimodal machine learning (ML): The present invention features ML techniques that process and pre-process educational data occurring in multiple modalities.

The strengths and weaknesses of the present invention are compared with the existing works in AI for special education and are highlighted in Table 1 below.

TABLE 1

| | Strengths | Weaknesses |
|---|---|---|
| Individualization | Individualized AI models | Unless precautionary ML |
| | can learn from the | techniques are employed, |
| | specific characteristics of | personalized models require |
| | an individual SEN | access to prior SEN data on |
| | student's data to adapt | every single student, such |
| | the educational content | as their level of |
| | based on the student's | performance, clinical and |
| | individualized education | academic history, and |
| | plans. | family background. These |
| | Individualized PM can | data may not be available, |
| | inform ambient learning | or the dataset is too small |
| | environments for | for conventional ML |
| | individual SEN students. | training. |
| | This improves the quality | |
| | of teaching and learning. | |
| Multimodal | Multimodal ML models | Domain expertise is |
| machine learning | can benefit from the | required to identify the |
| (ML) | inclusion of theory- | theory-grounded variables |
| | grounded variables, | and the corresponding data- |
| | especially when the | capturing methodology. |
| | theory provides a strong | Interdisciplinary effort is |
| | basis for explaining the | necessary for translating the |
| | relationship between | identified multimodal |
| | the variables and the | factors into multi-modal |
| | outcome. | variables and data. |
| | By combining | Advanced ML techniques |
| | educational data from | and models are required to |
| | multiple sources and | pre-process and process |
| | formats, multimodal PM | data existing in multiple |
| | can provide a more | modalities. |
| | holistic view of SEN | |
| | students' situation and | |
| | improve the accuracy of | |
| | predictions | |

Non-trivial solutions addressing the challenges that arise will be presented in subsequent sections.

### Problem Formulation

Multimodal and Individualized Predictive Model (MIPM) is further defined as a classifier that takes personalized categorical data and multimodal continuous data as input and outputs a classification target for an individual who is represented by the personalized data.

The three building blocks of the MIPM are further discussed below:

### Building Block 1: Multimodal Data Collection

Individualized SEN Data: The SEN data are those categorical variables that uniquely describe a student and his or her special needs. They include the School and Student identifiers (distinct integer values representing the participants uniquely and anonymously) and the Learning Task identifier (integer values representing the behavioral training task received by the student).

Classroom Environment Data: The classroom environment data includes the carbon dioxide concentration (CO₂ level), relative humidity (humidity), indoor temperature (temperature), and light intensity (light intensity). These data are collected by a set of environmental sensors installed in an IoT sensor box tailor-made for the current invention (FIG. 3). All measurements are made at 1 Hz.

Student Physiological Data: The physiological data are detected and collected from the Empatica E4 wristband, which are continuous time-varying series respectively measuring blood volume pulse (BVP) at 64 Hz, galvanic skin response (GSR) at 4 Hz, and the user's skin temperature (Skin Temp.) at 4 Hz. Furthermore, Inter-Beat Intervals (IBI) time values are also derived from the BVP signals.

Student Motion Data: The motion data captures the student's wristband-wearing hand's movement in the left (+ve) and right (−ve), up (+ve) and down (−ve), and front (+ve) and rear (−ve) directions in parallel. They are respectively named Aₓ, A_{y}, and A_{z} and are recorded at 32 Hz.

The total acceleration A is also derived by the following equation:

$$A = \sqrt{A {x}^{2} + {A}_{Y} 2 + A {z}^{2}} \qquad ( 1 )$$

Assessment Markers: A conventional assessment criteria in ABA is adopted as an assessment scale. In particular, each behavior response observed is assessed as plus (“+”), minus (“−”), prompt (“P”), or off task (“OT”). These assessment markers are input by human experts based on their subjective judgment. The outcome variable [symbol] is a binary variable computed from the ABA markers in a one-to-one correspondence manner. It indicates whether an expected behavior response is observed in the student where:

$$= \{ \begin{matrix}1 & if assessment marker equals “ + ” \\ 0 & otherwise\end{matrix} \qquad ( 2 )$$

### Building Block 2: Multimodal Data Fusion

Multimodal Translation: Inter-modal data translation is performed by exploiting the temporal information in existing data. Specifically, a multi-modality and multi-temporal sensing dataset is created, containing time instances of the same student's sensor data collected in the same experiment session to allow the prediction of missing sensor data values within a session.

Multimodal Data Alignment: An explicit alignment approach is utilized and temporal alignment is performed by algorithmically aligning the data using the timestamps associated with each modality. In this way, unimodal measurements can be synchronized and used the resulting multimodal data to produce a single, coherent dataset for further analyses.

Multimodal Representation: The data representations are created by the joint representation approach, where the unimodal representations are projected together into a multimodal space. Specifically, the joint representation is expressed as:

$$x = h ( {x}_{1} , {x}_{2} , … , {x}_{n} ) \qquad ( 3 )$$

Where h denotes the hidden layers of a deep neural network (DNN), x₁, x₂, . . . xₙ are the unimodal representations, and x is the resulting multimodal joint representation. Meanwhile, x is the penultimate layer of the DNN such that:

$$= f ( x ) ) \qquad ( 4 )$$

The output activation function f maps the penultimate layer x to the output y. The same DNN is used for both multimodal representation and fusion so that both components can be trained in an end-to-end manner.

### Building Block 3: MIPM Machine Learning

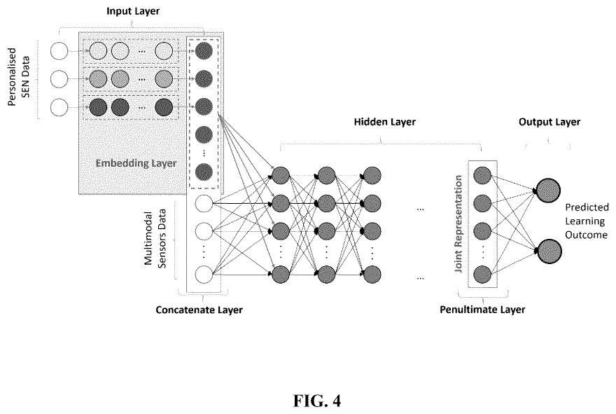

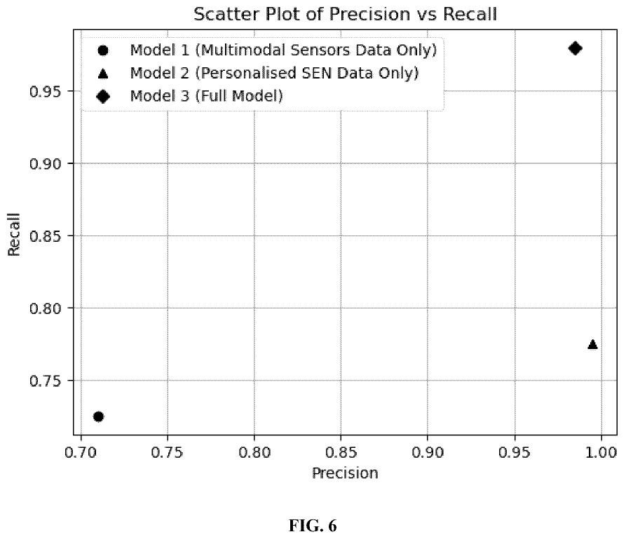

This building block consists of ML procedures in generic practices. Standard methods, e.g., training, cross-validation, and testing, are used to produce the model. The overall neural network architecture of MIPM is provided in FIG. 4. The corresponding prediction algorithm is also given.

Another embodiment of the present invention is a method of predicting a certain event for an SEN student using a theory-grounded artificial intelligence (AI) with personalized multimodal predictive modelling MIPM, wherein, the method comprising of:
1. capturing student's personal information, school information, learning task information, environmental sensor data, and physiological sensor data;
2. initiating the MIPM;
3. formulating an SEN embedding vector from the student information, the school information, and the learning task information;
4. formulating an input vector from the SEN embedding vector, the environmental sensor data and physiological sensor data;
5. training samples from the data collected in 1 through a random selection;
6. computing a loss using the MIPM and a training batch;
7. updating the MIPM parameters by minimizing the loss;
8. repeating steps 5 to 7 until converge or a preset upper limit of loops number is reached;
9. formulating a joint data representation vector from a real-time measurement; and,
10. making the prediction based on an optimized MIPM and the joint data representation vector.

Described below is an embodiment of the present invention employing the concept for the application for a more intuitive SEN learning.

### Example 1

### Data Collection

Empirical experiments are conducted using the present invention to collect authentic data for analyses. The experiments are held in ABA therapy sessions carried out between a student and a therapist. Each session involved at least one behaviour task in (1) Academic and Learning (AL), (2) Behaviour Development (BD), (3) Communication (CO), (4) Independence and Self-help (IS), (5) Sensory-Motor skills (SM), or (6) Socio-Emotional skills (SE). In this way, a dataset can be obtained from ABA experiment sessions involving various behaviour tasks.

### Participants and Procedures

The participants can be students diagnosed with SEN, such as mild to moderate ASD and/or intellectual disabilities (ID) with written consent from every participant's parent or guardian obtained before commencement. The steps below are performed using the present invention.

Algorithm 1: MIPM Algorithm

| Input | : | Training dataset D = {Students υ, Schools σ, Tasks τ, sensors data |
|---|---|---|
| | | (s₁, s₂, ..., sₙ), labels y}, test data υ′, σ′, τ′, (s′₁, s′₂, ..., s′ₙ), initial |
| | | model M₀. |
| Output | : | Predicted ABA marker y′ ∈ {0, 1} |

Model Building:
foreach d ∈ D do

| | |
|---|---|
| \| | Formulate SEN embedding vector: |
| \| | ρ = υ\| \| σ\| \| τ |
| \| | Formulate input vector: |
| \| | v = (ρ, s₁, s₂, ..., sₙ) |

```
end
for k = 0, 1, 2, ... do
  |
  |
  |
```

Randomly select training samples from D;
Compute the loss using the current model Mₖ and the training batch;
Update the model parameters for Mₖ₊₁ by minimizing the loss;

end
begin

| | |
|---|---|
| \| | Formulate joint representation: |
| \| | ρ′ = υ′\| \| σ′\| \| τ′; |
| \| | v′ = (ρ′, s′₁, s′₂, ..., s′ₙ); |
| \| | x′ = h(v′) |
| \| | Make prediction: |
| \| | y′ = M(x′) |
| end | |

1. A target behaviour and its component tasks are retrieved from the system.
2. The therapist teaches the component tasks by:
   introduces one or more stimuli, and
   if necessary, provides a prompt (e.g., gestural guidance) to facilitate the student's correct response.
3. The therapist inputs an assessment marker to the system and continues or pauses the training according to the student's condition reflected in the sensor readings.
4. Repeat steps 2 and 3 until the student gives the correct response or the session ends.

A timestamp is automatically added to the therapist's input in step 3 above. In this way, multimodal sensor data and the assessment markers are gathered by the present invention in real-time.

### Measurements

Motivated by reproducibility in behaviour-based intervention settings, participants' learning performance is measured by the following score:

$$\frac{\# “ + ” behavior responses}{\# behavioral tasks performed in the session} \times 100 \% \qquad ( 5 )$$

where the behavior response assessment is made by the therapist conducting the session. A follow-up probe test is conducted six months later to determine whether the learned behavior is maintained. The criterion of task mastery is defined as having the probe performance score higher than or equal to that obtained in the training session. Around a quarter (25%) of the tasks will be randomly selected and reviewed by another therapist who is not involved in the training. The Cronbach's a value can be calculated to verify interrater reliability.

Since the ML problem is a binary classification problem, MIPM model's predictive performance can be evaluated using the ML metrics applicable to binary classifiers. Namely, accuracy, precision, recall, and F1 score. In particular, F1 score is the harmonic mean of precision and recall; where

$$F 1 score = \frac{2 \times ( precision \times recall )}{( precision + recall )} \qquad ( 6 )$$

### Results in Example 1

### Baseline and Full Models

The models with (1) multimodal sensor data predictors (Model 1), personalised SEN data predictors (Model 2), and both sensor and SEN data predictors (Model 3) are studied (Table 2). The three models are compared using the Akaike information criterion (AIC). The full model (Model 3) significantly outperforms the two baseline models with either only sensor data predictors (Model 1) (Δx2=2142, Δdf=11, p<0.001) or SEN data predictors (Model 2) (Δx2=98, Δdf=3, p<0.001) alone. Also, the full model yields the lowest BIC among all models. The full model is adopted in subsequent ML training and evaluation.

TABLE 2

| | Model 1 | Model 2 | Model 3 |
|---|---|---|---|
| | B (SE) | B (SE) | B (SE) |
| Sensor Data | | | |
| CO₂ Level | 0.01(0.01) | — | 0.09(0.10)*** |
| Humidity | 0.03(0.01)* | — | 0.01(0.01) |
| Temperature | −0.01(0.01) | — | 0.02(0.06) |
| Light Intensity | −0.03(0.01)* | — | 0.01(0.01) |
| IBI | 0.06(0.01)*** | — | 0.05(0.01)*** |
| GSR | −0.01(0.01) | — | −0.04(0.01)*** |
| Skin Temp. | −0.00(0.01) | — | 0.01(0.01) |
| Acc. X Orien. | −0.03(0.01)* | — | −0.00(0.01) |
| Acc. Y Orien. | 0.02(0.01) | — | 0.01(0.01) |
| Acc. Z Orien. | 0.01(0.01) | — | 0.01(0.01) |
| Total Acc. | −0.00(0.01) | — | 0.01(0.01) |
| SEN Data | | | |
| School | — | 0.38(0.03)*** | 0.41(0.03)*** |
| Student | — | −0.01(0.00)$** | −0.01(0.00)* |
| Learning Task | — | 0.00(0.00)* | 0.00(0.00) |
| Model | | | |
| Summary | | | |
| Df Model | 3 | 11 | 14 |
| Adjusted R² | 0.014 | 0.487 | 0.505 |
| Log-Likelihood | −3302.7 | −2287.0 | −2228.4 |
| AIC | 6627 | 4583 | 4485 |
| BIC | 6694 | 4602 | 4576 |

*p < .05,
**p < .01,
***p < .001
Dependent variable: Behavior Response ∈ {0, 1}

### Performance Evaluation

The model's performance is evaluated in two ways. First, the neural networks are constructed for Models 1 to 3 and obtained the confusion matrix for each of the models. Next, the selected model's performance is benchmarked with a few existing results reported in the field.

The neural network presented in FIG. 4 is built using TensorFlow 2.11.0 and ran it on an NVIDIA RTX A2000 12 GB GPU). GridSearchCV of the Python scikit-learn 1.2.2 open-source library is used to tune the hyper-parameters of the network.

The hyperparameters are selected based on the F1 scores. In the end, a DNN is established with 158 input nodes (150 for personalized SEN categorical data and 8 for multimodal sensors continuous data) and two output nodes (for the two classes). The best-performed DNN model does not use any resampling, and the resulting DNN has seven hidden layers, with 64 nodes on the first hidden layer and 48 nodes on the remaining layers. Sigmoid is used as the activation function. The model is run for 20,000 epochs with an initial learning rate of 0.001, a batch size of 128, a dropout rate of 0.001, and a momentum of 0.92. The scatter plot of precision vs recall for the two baseline models and the full model is provided in FIG. 5. At the same time, the benchmarking results are provided in Table 3.

TABLE 3

| | Special | Prediction | | | | | | F1 |
|---|---|---|---|---|---|---|---|---|
| | Needs | Target | Personalization | Multimodality | Accuracy | Precision | Recall | Score |
| Previous | ASD | Engagement | ✓ | ✓ | 60.7% | — | — | — |
| work 1 | | level | | | | | | |
| Previous | ASD | Emotion | ✓ | — | 74.7% | — | — | — |
| work 2 | | | | | | | | |
| Previous | LD | Academic | ✓ | — | 97% | 98% | 97.5% | 97% |
| work 3 | | performance | | | | | | |
| Previous | Deafness | Sign | — | ✓ | 90.00% | 90.00% | 89.90% | 90.56% |
| work 4 | | language | | | | | | |
| The present | ASD, ID | Positive | ✓ | ✓ | 98.18% | 97.86% | 98.49% | 98.17% |
| invention | | behaviours | | | | | | |

### Discussions Based on the Results in Example 1

Data Collection: The known factors affecting SEN students' learning are reviewed. In addition to collecting personalized SEN data and ABA markers, an IoT sensor box (FIG. 3) is developed to collect ambient environmental data, including CO₂ level, humidity, temperature, and light intensity from special education classrooms.

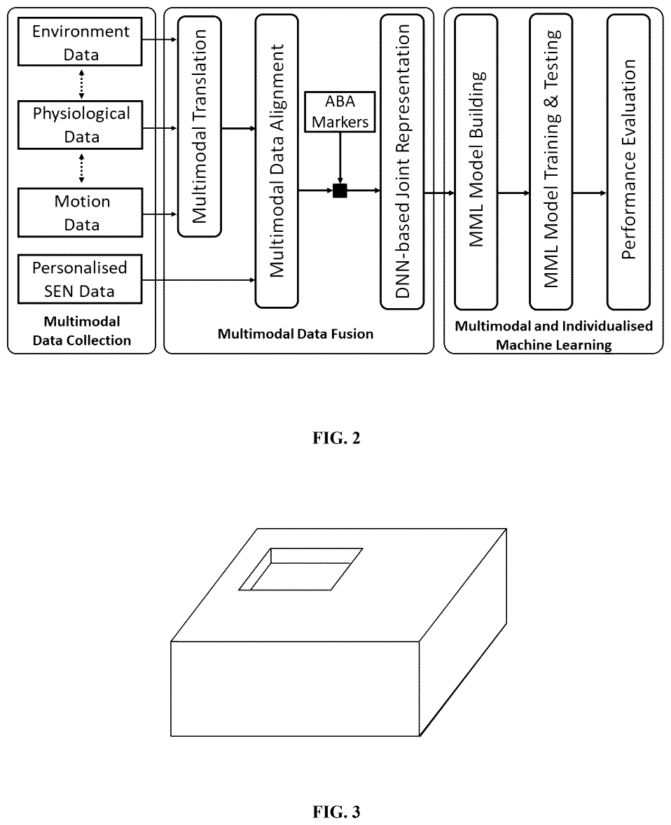

Problem Modelling: The MIPM framework (FIG. 2) is defined as a theory-grounded AI approach to individualized learning for students with SEN. The model is supported by a solid theoretical foundation in special education. Multimodal ML techniques, including multimodal data collection, fusion, and joint representation, are implemented presently.

Field Tests and Evaluation: The empirical evaluation results show that MIPM significantly improved over baseline models having either SEN data or sensor data only. Furthermore, the full model can achieve predictive performance metrics comparable with the few reported results in the field (Table 3). Based on the results obtained, the inventors have established two theories below:

Theorem 1 (Individualization of Predictive Models). The inclusion of theory-grounded individualized data can significantly improve the performance of a predictive model.

Theorem 2 (Data Multi-modality). The inclusion of theory-grounded multimodal sensor data can significantly improve the performance of a predictive model.

The current prediction target is binary (either plus (“+”) or not) and limits the ABA outcomes interpretation. Alternatively, the number of classifier outputs may further include but not limited to three other outcomes namely, minus (“−”), prompt (“P”), and off task (“OT”).

Alternatively, the present invention may be utilized in a daily classroom environment wherein the classroom may have one-to-a-few or one-to-many teachers to student(s) ratio.

In view of the above, the present invention is more advantageous over existing technologies as the technology offers personalization with real-time processing and data analysis. Therefore, the results obtained are highly accurate and very individualized towards an SEN student.

The present invention explained above is not limited to the aforementioned embodiment and drawings, and it will be obvious to those having an ordinary skill in the art of the present invention that various replacements, deformations, and changes may be made without departing from the scope of the invention.

1. A system for predicting behavioral performance of a special-need student using artificial intelligence comprising:
one or more processors; and
a cloud server coupled to the one or more processors, wherein the cloud server comprises a plurality of building blocks comprising:
a first building block configured to receive raw multimodal data from a plurality of sensing devices;
a second building block configured to pre-process the collected multimodal data and produce a joint data representation vector over a defined time window;
a third building block configured to predict the performance of the special-need student using an optimized machine learning module; and
wherein, the plurality of building blocks is in the form of programmable instructions executable by the one or more processors.

2. The system according to claim 1, wherein the first building block is a multimodal data collection module.

3. The system according to claim 1, wherein the second building block is a multimodal data fusion module.

4. The system according to claim 3, wherein the multimodal data fusion module further comprises:
a multimodal translation module that employs temporal information in the multimodal raw data to translate and predict missing data values;
a multimodal data alignment module that performs temporal alignment and algorithmically aligns the translated data based on the defined time window to produce a unified dataset; and
a deep neural network (DNN) joint representation module that employs the unified dataset to produce a joint data representation vector for subsequent analysis.

5. The system according to claim 1, wherein the third building block is a machine learning module configured to train, cross-validate, test and predict the behavioral performance of the special-need student.

6. The system according to claim 1, wherein the collected multimodal data are student's individualized categorical variables that uniquely couple with student special needs (SEN) data, classroom environment data, physiological data, and motion data.

7. The system according to claim 1, wherein the plurality of the sensing devices comprises:
an IoT sensor box; and
a plurality of sensors in the IoT sensor box, and the plurality of sensors include a temperature sensor, a humidity sensor, and a CO₂ sensor;
each of the plurality of sensors is operatively connected to the IoT sensor box to transmit data to the second building block.

8. The system according to claim 7, wherein the plurality of the sensing devices further comprises wearable sensors configured to measure heart rate, sweat, and motion.

9. The system according to claim 4, wherein the unified dataset permits alignment and fusion of multimodal data across different time windows.

10. The system according to claim 1, wherein the one or more processors is at least one computing device with internet access, which includes but is not limited to an Edge PC, a tablet, and the like.

11. The system according to claim 1, wherein the cloud server is configured to store and process multimodal data for model refinement and continuous learning through a feedback module.

12. A method for predicting behavioral performance of a special-need student using artificial intelligence, the method comprising:
collecting real-time multimodal data;
pre-processing the collected real-time multimodal data via a multimodal data fusion module and producing a joint data representation vector over a defined time window; and
predicting the behavioral performance of the special-need student using a machine learning module.

13. The method according to claim 12, wherein the step of collecting the real-time multimodal data further comprises:
placing a plurality of sensing devices to the student and the student's classroom environment;
capturing the student's classroom environment data, physiological data, and motion data; and
transmitting the real-time multimodal data via a wireless connection to a multimodal data collection module.

14. The method according to claim 12, wherein the step of pre-processing the collected multimodal data further comprises:
retrieving existing SEN data;
translating the captured data via a multimodal translation module;
formulating an embedding vector for the SEN data;
aligning the multimodal data to ensure timestamps consistency between each data collected via a multimodal data alignment module;
providing at least one stimulus and prompt using an assessment marker by a human expert via a computing device and automatically tagging a timestamp to each data inputted by the human expert;
formulating an input vector by combining the embedding vector for the SEN data and translated data; and
applying a deep neural network (DNN) to the input vector, producing the joint data representation vector and projecting into a multimodal space for subsequent analysis.

15. The method according to claim 12, wherein, the step of predicting the behavioral performance of the special-need student in real-time using the machine learning module further comprises:
feeding the predicted behavioral performance back to the machine learning module via a feedback module.

16. The method according to claim 12, wherein the step of translating the captured data via a multimodal translation module further comprises:
creating a multi-modality and multi-temporal sensing dataset;
containing time stances of the student's SEN data collected in a session; and
predicting missing sensor data in the session.

17. The method according to claim 12, wherein the step of aligning the multimodal data to ensure timestamps consistency between each data collected via a multimodal data alignment module further comprises:
synchronizing unimodal measurements; and
producing a single and coherent dataset.

18. The method according to claim 12, wherein the step of collecting the real-time multimodal data further comprises:
selecting at least one SEN data from a group of behavioral tasks including academic and learning tasks, behavior development, communication, independence and self-help, sensory-motor skills and socio-emotional skills.

19. The method according to claim 12, wherein, the step of collecting the real-time multimodal data further comprises selecting the student being diagnosed with learning disabilities including but not limited to mild to moderate autism spectrum disorder (ASD) or intellectual disabilities.

20. The method according to claim 14, wherein the step of providing at least one stimulus and prompt using the assessment marker by the human expert via the computing device and automatically tagging the timestamp to each data inputted by the human expert further comprises:
continuing or pausing the step of providing the at least one stimulus and the prompt according to a student's condition based on the data captured by the plurality of the sensing devices; and
repeating the step of providing at least one stimulus and the prompt until the student provides a correct response or session ends.

21. The method according to claim 14, wherein the step of applying the deep neural network (DNN) to the input vector, producing the joint data representation vector, and projecting into the multimodal space for subsequent analysis by the machine learning module further comprises:
processing the input vector through hidden layers of the DNN;
utilizing a penultimate layer of the DNN; and,
applying an output activation function to the penultimate layer to map the joint to output the vector.

22. The method according to claim 18, wherein, the step of predicting the behavioral performance of the special-need student in real-time using the machine learning module further comprises:
utilizing the joint data representation vector to perform the behavioral prediction of the performance of the special-need student.

23. A method of training and optimizing a machine learning module for predicting behavioral performance of a special-need student comprising:
(a) collecting multimodal data via a multimodal data collection module as a training dataset;
(b) storing the training dataset in a cloud server;
(c) initiating the machine learning module;
(d) selecting random samples from the training dataset;
(e) computing data loss and updating the machine learning module's parameters by minimizing the data loss; and
(f) repeating steps (d)-(e) until convergence is achieved or a pre-determined upper limit of loops number is reached.

24. The method according to claim 23, wherein the method further comprises:
formulating a joint data representation vector from a real-time measurement; and,
making a prediction.
